(19) United States
(12) Patent Application Publication
(10) Pub. No.: US 2017/0311899 A1
(43) Pub. Date: Nov. 2, 2017

(54) APPARATUS AND METHOD FOR IDENTIFYING MOVEMENT IN A PATIENT

(71) Applicant: Mayo Foundation for Medical Education and Research, Rochester, MN (US)

(72) Inventors: Kenton R. Kaufman, Rochester, MN (US); Melissa M. Morrow, Rochester, MN (US); Vipul A. Lugade, Rochester, MN (US); Emma Fortune, Rochester, MN (US)

(21) Appl. No.: 15/647,574

(22) Filed: Jul. 12, 2017

Related U.S. Application Data
(63) Continuation of application No. 14/338,414, filed on Jul. 23, 2014, now abandoned.
(60) Provisional application No. 61/857,630, filed on Jul. 23, 2013.
(60) Provisional application No. 61/857,892, filed on Jul. 24, 2013.

(51) Int. Cl. A61B 5/00; A61B 5/11

(52) U.S. Cl. CPC A61B 5/726; A61B 5/6801; A61B 5/112; A61B 5/1118

(57) ABSTRACT

Methods for operating a processing system to generate accurate information representative of movement of a body from activity sensors such as tri-axial accelerometers. The system uses wavelet analysis and/or adaptive thresholds to provide quantitative measurements of a person's posture and/or activity, including low-speed activity, during daily living.

### CROSS REFERENCE TO RELATED APPLICATIONS

This application is a continuation of U.S. patent application Ser. No. 14/338,414, filed Jul. 23, 2014, entitled APPARATUS AND METHOD FOR IDENTIFYING MOVEMENT IN A PATIENT, which claims the benefit of U.S. Provisional Patent Application No. 61/857,630, filed Jul. 23, 2013, entitled APPARATUS AND METHOD FOR IDENTIFYING MOVEMENT IN A PATIENT, and U.S. Provisional Patent Application No. 61/857,892, filed Jul. 24, 2013, entitled APPARATUS AND METHOD FOR IDENTIFYING MOVEMENT IN A PATIENT, which applications are incorporated herein by reference in their entirety and for all purposes.

### GOVERNMENT LICENSE RIGHTS

This invention was made with government support under HD007447 and HD065987 awarded by the National Institutes of Health and W81XWH-11-2-0058 awarded by the U.S. Army. The government has certain rights in the invention.

### APPENDICES

This application includes the following appendices after the claims. These appendices are incorporated herein by reference for all purposes:

1. Appendix A entitled “Validity of Using Tri-Axial Accelerometers to Measure Human Movement-Part I: Posture and Movement Detection,” and

2. Appendix B entitled “Validity of Using Tri-Axial Accelerometers to Measure Human Movement-Part II: Step Counts at a Wide Range of Gait Velocities.”

### FIELD OF THE INVENTION

The invention relates generally to apparatus and methods for indentifying movement in a body, such as physical activity in a human patient.

### BACKGROUND

Devices and methods for identifying and quantifying body position and movement are generally known. There remains, however, a continuing need for improved devices of these types. In particular, there is a need for apparatus and methods capable of accurately identifying relatively slow speed movement.

### SUMMARY

Embodiments of the invention include methods for operating a processing system to generate accurate information representative of movement of a body. On embodiment includes receiving one or more kinematic or movement signals representative of movement of the body at the processing system, continuous wavelet transform processing the one or more movement signals by the processing system to generate continuous wavelet transform data, and determining by the processing system whether the body is moving as a function of the continuous wavelet transform data. Another embodiment includes receiving one or more kinematic or movement signals representative of movement of the body at the processing system, processing the movement signals by the processing system to generate one or more step threshold levels representative of steps, and processing the movement signals by the processing system, including comparing the movement signals to the one or more step threshold levels, to identify patient steps.

### BRIEF DESCRIPTION OF THE DRAWINGS

FIG. 1 is a block diagram of a body movement identifying system in accordance with embodiments of the invention.

### DESCRIPTION OF THE INVENTION

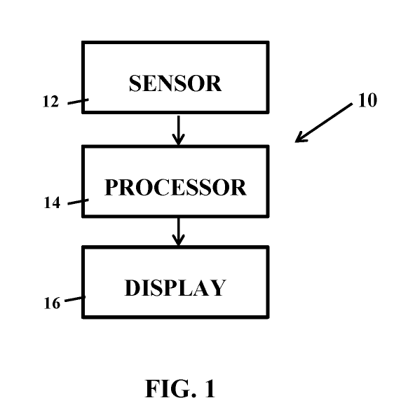

FIG. 1 is an illustration of a body movement identifying system 10 in accordance with embodiments of the invention. System 10 can objectively and accurately measure physical activity, such as movement or steps of a human patient or other body, including when the body is moving relatively slowly (e.g., less than 2 m/sec.). As shown, system 10 includes a sensor 12, processor 14 and display 16. Sensor 12 can, for example, include one or more tri-axial or other accelerometer-type sensors mounted to the body being evaluated (i.e., body-worn sensors), and provide accelerometer or other signals representative of kinematics or movement of the body. Other embodiments of the invention include other sensors for providing the kinematic signals, such as gyroscopes and magnetometers. One or more sensors 12 can be mounted to any portion of the body at which they will provide kinematic signals representative of movement, including the waist and lower extremities such as the ankle. Processor 14 can be a programmed computer including non-transitory memory having stored instructions for processing accelerometer or other movement signals received from the sensor 12. In other embodiments the processor 14 can be configured as a dedicated or application-specific device, or in other forms to provide the functionality described herein. Processor 14 can also include memory (not shown) for storing the identified movement data or information (e.g., identified movement episodes, the speed or category (e.g., walking or jogging) of the movement, and the number of identified steps. The identified movement data can be displayed on display 16. By way of non-limiting examples, system 10 can be configured as described in the following papers that are attached hereto as Appendices A and B and incorporated herein by reference: (1) Validity of using tri-axial accelerometers to measure human movement-Part I: Posture and movement detection, and (2) Validity of Using Tri-Axial Accelerometers to Measure Human Movement-Part II: Step Counts at a Wide Range of Gait Velocities.

In accordance with one embodiment of the invention, accurate detection of postural transitions, walking, and jogging is determined from body accelerations using continuous wavelet transforms. Using continuous wavelet transform processing, it is possible to determine the changing frequency content over time on a non-stationary signal. By representing the signal as a sum of a scaled and time shifted mother wavelet, continuous wavelet transforms can provide utility in obtaining transition and gait pattern information. Continuous wavelet transforms (CWT) enhance the ability of system 10 to identify movement at all speeds, including slow walking instants (e.g., speeds less than about 1.0 m/sec.).

The gravitational and bodily motion components of the acceleration or other kinematic or movement signal are used to identify all possible outcome configurations. The bodily motion component was utilized in determining static versus dynamic activity, with signal magnitude area (SMA) values above a first threshold level (e.g., 0.135 g) identified as being representative of movement. The signal magnitude area was computed over each 1 sec. window (t) across all three orthogonal axes (aₓ, a_{y}, a_{z}).

SMA=1/t×(∫aₓ(t)dt+∫a_{y}(t)dt+∫a_{z}(t)dt)

Of those seconds of data identified as non-movement (e.g., those seconds below the first threshold level), a continuous wavelet transform was utilized to process the movement signals. The Daubechies 4 Mother Wavelet algorithm was applied to data received from a waist sensor in one embodiment of the invention. Other algorithms and movement signals can be used in other embodiments. Data which fell within a predetermined frequency range (e.g., 0.1-2.0 Hz) was further identified as movement. In other embodiments, movement is identified by evaluating whether the scaling value exceeds a threshold (e.g., 1.5) over a predetermined time period (e.g., about 1 sec.). In still other embodiments, movement is identified when the data content meets both the frequency and scaling value criteria.

In another embodiment of the invention, which can be implemented alone or in combination with the continuous wavelet transform embodiment described above, patient steps can be accurately identified and counted at all speeds, including at relatively slow speeds, in accordance with an adaptive thresholding algorithm. During identified walking and jogging movement segments, the anteroposterior accelerations or other movement signals from sensors 12 such as, for example, those on the right and left ankles, can be filtered (e.g., using a low-pass butterworth filter with a cut-off frequency of 6 Hz) and analyzed using a peak detection method with adaptive thresholds to calculate the number of steps taken. The adaptive thresholds for peak detection allow for a greater accuracy in the detection of steps at different walking speeds. For each continuous segment of data classified as walking or jogging, adaptive thresholds to detect heel-strike points were calculated, and optionally periodically updated. Local minimum peaks of the anteroposterior acceleration signal (αAP) were considered valid heel strike points (e.g., measurement signals determined or identified as being representative of steps) if their magnitudes were greater than a first step threshold value or level. In embodiments, the first threshold (e.g., th₁ below) can be the mean of the anteroposterior acceleration signal (α̅_{AP}) and determined as a function of the number N is the number of samples.

th₁=0.8×(1/N)×Σᵢ₌₁^{N}(α_{AP}_{ᵢ}>α̅_{AP})

In other embodiments, valid heel strike points (i.e., given movement signal portions) are determined as a function of a second step threshold level if the movement signal representative of a previous step had a preceding maximum whose magnitude is greater than the second step level threshold, where the second threshold level is greater by a predetermined value or amount (e.g., th₂ below) than the first step threshold.

th₂=0.6×max(α_{AP})

Still other embodiments identify steps using both the first and second threshold levels (i.e., local minimum peaks of the given anteroposterior acceleration signal). Heel strike points are considered valid if their magnitudes were greater than the first step threshold level and had a preceding maximum whose magnitude was at least the predetermined amount greater than the minimum.

In addition to adaptive acceleration thresholds, adaptive timing thresholds can also be calculated and used. If two minimum peaks are found within a first (e.g., variable or adaptive) step time threshold (i.e., t₁ below) of each other for walking and a second (e.g., predetermined or fixed) step time threshold such as 0.25 sec. of each other for jogging, only the one of greater amplitude may be considered as a heel-strike point.

t₁=fₛ×0.1/mean(SMA)

The first timing threshold can be calculated for each walking activity segment as a function of the sampling frequency (fₛ) and the signal magnitude area SMA. A minimum value such as 0.5 sec. can be set for the first timing threshold.

To enhance the ability to address this issue of activity with high variability of heel strike accelerations (particularly during walking segments which included stair climbing), the algorithm can be extended to check for missing steps in each segment of data by calculating the difference in time between each successive identified heel-strike point. For walking (i.e., a first speed category), if there was a first time interval such as 2.5 sec. or longer between successive heel-strike points (2.0 sec. or longer between the first heel-strike point and the start of the activity segment and the last heel-strike point and the end of the activity segment), the acceleration thresholds were updated or recalculated for the segment of data within 0.5 sec. from either heel-strike point and new heel strike points were looked for within that segment. For jogging (i.e., a second speed category) if the time interval was a second time interval such as 1.25 sec. or longer between successive heel-strike points (1 sec. or longer between the first heel-strike point and the start of the activity segment and the last heel-strike point and the end of the activity segment), the acceleration thresholds were recalculated or updated for the segment of data within 0.25 sec. from either heel-strike point and new heel-strike points were sought within that segment.

Although the present invention has been described with reference to preferred embodiments, those skilled in the art will recognize that changes can be made in form and detail without departing from the spirit and scope of the invention. In particular, the continuous wavelet transform algorithm and the adaptive threshold step counting algorithm can be used alone or in combination, and either or both algorithms can be used in combination with other movement detection algorithms such as, for example, those in the articles identified above and incorporated herein.

1. A method for operating a processing system to generate information representative of movement of a body, comprising:
receiving one or more kinematic or movement signals representative of movement of the body at the processing system;
continuous wavelet transform processing the one or more movement signals by the processing system to generate continuous wavelet transform data; and
determining, by the processing system, whether the body is moving as a function of the continuous wavelet transform data.

2. The method of claim 1 wherein determining whether the body is moving includes determining whether the body is moving at relatively slow speeds, optionally including or consisting of speeds between about 0.10-1.0 m/sec.

3. The method of claim 1 wherein determining whether the body is moving as a function of the wavelet transform data includes:
processing the wavelet transform data to identify frequency content in the wavelength transform data; and
determining whether the body is moving as a function of the identified frequency content.

4. The method of claim 3 wherein determining whether the body is moving as a function of the identified frequency content includes determining whether the identified frequency content is within a predetermined frequency range, optionally including or consisting or frequencies between about 0.1-2.0 Hz.

5. The method of claim 1 wherein determining whether the body is moving as a function of the wavelet transform data includes:
processing the wavelet transform data to identify a scaling value in the wavelength transform data; and
determining whether the body is moving as a function of the identified scaling value.

6. The method of claim 5 wherein determining whether the body is moving as a function of the identified scaling value includes determining whether the identified scaling value exceeds a predetermined threshold, optionally including a threshold value of about 1.5, over a predetermined time period, optionally including a time period of about 1 sec.

7. The method of claim 1 wherein:
the method further includes signal magnitude area processing the one or more movement signals to generate signal magnitude data; and
determining whether the body is moving includes determining whether the body is moving as a function of the wavelet transform data and the signal magnitude data.

8. The method of claim 7 wherein determining whether the body is moving includes identifying movement of the body based on the waveform transform data when the signal magnitude data is representative of non-movement, optionally when the signal magnitude data has a value below a predetermined threshold value, optionally a threshold value of about 0.135 g.

9. The method of claim 1 wherein receiving one or more movement signals includes receiving one or a plurality of acceleration signals.

10. The method of claim 9 wherein each acceleration signal is produced by a sensor attached to the body.

11. The method of claim 1 wherein the continuous wavelet transform processing includes processing using a Daubechies 4 Mother Wavelet transform algorithm.

12. A method for operating a processing system to generate information representative of movement of a body, comprising:
receiving one or more kinematic or movement signals representative of movement of the body at the processing system;
processing the movement signals by the processing system to generate one or more step threshold levels representative of steps; and
processing the movement signals by the processing system, including comparing the movement signals to the one or more step threshold levels, to identify patient steps.

13. The method of claim 12 wherein receiving one or more movement signals includes receiving one or a plurality of acceleration signals.

14. The method of claim 13 wherein each acceleration signal is produced by a sensor attached to the body.

15. The method of claim 12 and further including periodically updating one or more of the step threshold levels.

16. The method of claim 12 wherein identifying patient steps includes identifying heel-strike points.

17. The method of claim 12 herein comparing the movement signals to the step threshold levels includes comparing local minimum peaks of the movement signals to the step threshold levels.

18. The method of claim 12 wherein comparing the movement signals to identify steps includes identifying a given movement signal as representative of a step if the given movement signal is greater than a first step threshold and a movement signal of a preceding indentified step is greater than a second threshold level, and wherein the second threshold level is optionally greater than the first threshold level.

19. The method of claim 12 wherein:
generating the step threshold levels includes generating a first step threshold level as a function of movement signals representative of a velocity of the body; and
comparing the movement signals includes comparing the movement signals to the first step threshold level.

20. The method of claim 12 wherein the movement signal is an anteroposterior acceleration signal.
